(19) United States
(12) Patent Application Publication
(10) Pub. No.: US 2006/0281801 A1
(43) Pub. Date: Dec. 14, 2006

(54) Process for the preparation of valsartan and its intermediates

(76) Inventors: Ashok Kumar, Mumbai (IN); Manmohan Madhavrao Nimbalkar, Mumbai (IN); Sanjay Govind Barve, Mumbai (IN); Dattatray Shamrao Metil, Thane (IN); Rahul Suresh Kelkar, Mumbai (IN); Bharat Dinkar Shimpukade, Pune (IN); Lavkesh Dayashankar Kushwaha, Thane (IN)

Correspondence Address: BLANK ROME LLP, 600 NEW HAMPSHIRE AVENUE, N.W., WASHINGTON, DC, 20037, US

(21) Appl. No.: 11/405,522

(22) Filed: Apr. 18, 2006

(30) Foreign Application Priority Data
Apr. 19, 2005 (IN) 490/MUM/2005

(51) Int. Cl. A61K 31/41; C07D 257/02

(52) U.S. Cl. 514/381; 548/253

(57) ABSTRACT

The present invention relates to an improved process for the preparation of valsartan and its intermediates in substantially pure enantiomeric form. In particular, the present invention provides a process for preparing benzyl valsartan intermediate substantially free of organotin impurities. The valsartan produced from such benzyl valsartan intermediate requires significantly lower catalyst loading and has superior purity.

This application claims the priority of Indian Patent Application No. 490/MUM/2005, filed Apr. 19, 2005, which is incorporated herein by reference.

### FIELD OF INVENTION

The present invention relates to an improved process for the preparation of a known pharmaceutical agent, valsartan, and its intermediates in substantially pure enantiomeric form.

### BACKGROUND OF THE INVENTION

(S)-N-(1-Carboxy-2-methyl-prop-1-yl)-N-pentanoyl-N-[2′-(1H-tetrazol-5 -yl)-biphenyl-4-ylmethyl]-amine commonly known as valsartan has the following structure (Formula I): 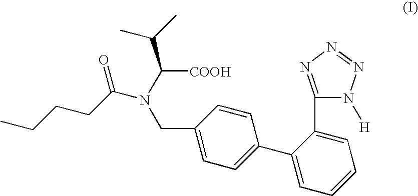
Valsartan is a member of the class of agents termed angiotensin-II (AT) receptor antagonists having effective anti-hypertensive activity with an excellent profile of safety and tolerability. Activation of AT receptors in the outer membrane of vascular smooth muscle cells of the heart and arteries causes the tissues to constrict. AT-I receptors are activated by an octa-peptide, angiotensin-II. Angiotensin-II helps to maintain constant blood pressure despite fluctuations in a person's state of hydration, sodium intake and other physiological variables. Angiotensin-II also performs the regulatory tasks of inhibiting excretion of sodium by the kidneys, inhibiting nor-ephedrin reuptake and stimulating aldosterone biosynthesis. By inhibiting the angiotensin-II to AT receptors binding, valsartan disrupts the vasoconstriction mediated by AT receptors.

Valsartan is therefore a non-peptide angiotensin-II antagonist, inhibiting the actions of angiotensin-II on its receptors , thus preventing the increase of blood pressure produced by the hormone-receptor interactions. Hence, it is used in the treatment of cardiovascular indications, such as hypertension and heart failure. Comparative trial studies have shown that valsartan is as effective as angiotensin-converting enzyme (ACE) inhibitors, calcium-channel blockers, and α-blockers, and is generally better tolerated. Valsartan is marketed as the free acid under the trade name DIOVAN, however, its combination with diuretics, such as hydrochlorothiazide, have specific advantage as anti-hypertensive agent.

The synthesis of Valsartan and its intermediates of formula II, III and IV are reported in U.S. Pat. No. 5,399,578 (the '578 patent) and Bioorganic & Medicinal Chemistry Letters, vol. 4, pp 29-34, 1994, via the following method as shown in Scheme 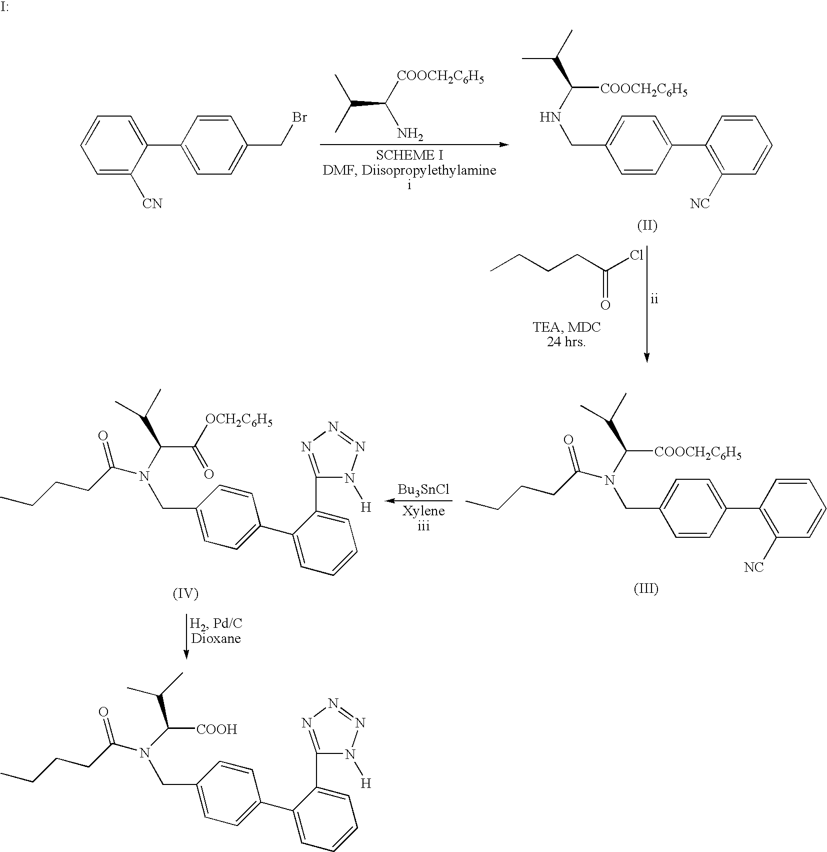
This process appears in some respects to be practical but involves many disadvantages from the point of view of purity/yield of valsartan and its intermediates. The major problems are incomplete reactions, long reaction times, contamination of valsartan with a number of impurities or starting material/intermediates, and lower chiral purity of valsartan obtained.

Particular disadvantages of synthesis disclosed in U.S. Pat. No. 5,399,578 include long reaction time and incomplete reaction of (S)-N-[(2′-cyanobiphenyl-4-yl)methyl]-(L)-valine benzyl ester (Formula II) or salts thereof with valeroyl chloride in chlorinated solvents in the preparation of (S)-N-[(2′-cyanobiphenyl-4-yl)methyl]-N-valeroyl-(L)-valine benzyl ester (Formula III). This can result in contamination of compound HI. This impurity in turn is carried forward in subsequent reaction steps and results in valsartan of low purity.

A further disadvantage of prior method is contamination of large amounts of various impurities especially organgtin impurity in the penultimate intermediate of valsartan namely, (S)-N-[(2′-(1H-tetrazol-5-yl)biphenyl-4-yl)methyl]-N-valeroyl-(L)-valine benzyl ester of the formula IV (benzyl valsartan), during the tetrazole formation of Formula III. An effective purification or means to remove the organotin by-product and other impurities from benzyl valsartan are not described or disclosed in '578 patent. As a result, the catalyst (palladium-charcoal) gets poisoned in the presence of organotin impurity during the debenzylation of benzyl valsartan (Formula IV) to valsartan, and necessitates a very high loading of palladium-charcoal for completing the reaction. This is coupled with susceptibility of valsartan or its intermediate to partial racemization during the reaction conditions or purification processes as described in '578 patent. This makes the prior art process impractical for obtaining a high purity valsartan for clinical use. Racemization herein means the process of a relatively pure enantiomer of a substance becoming a mixture of enantiomeric forms. Valsartan is used in the enantiomerically pure (S)-N-[(2′-(1H-tetrazol-5-yl)biphenyl-4-yl)methyl]-N-valeroyl-(L)-valine form in pharmaceutical compositions. This prevents a synthetic chemist from using drastic conditions to push the reactions for completion when reactions are slow, and poses considerable challenges to design milder reaction conditions

Conventional processes generally employ solvents like Dimethyl formamide (DMF) during the preparation of compound of formula II and formula III and there is a current need to avoid or minimize the use of such environmentally friendly materials.

Thus there is a need in the art for an improved widi cost-effective synthetic process for the preparation of valsartan and precursors thereof in substantially enantiomerically pure form.

### SUMMARY OF THE INVENTION

It is an objective of the present invention to overcome or ameliorate at least one of the disadvantages of the prior art or to provide a useful alternative.

It is an object of the present invention in its preferred form to provide an industrial process for the preparation of valsartan substantially in its pure enantiomeric form.

Other objectives of this invention include establishment of suitable purification methods to remove the organo-tin by-product from benizyl valsartan, to overcome the incomplete reactions during preparation of the intermediates compounds, to speed-up the reactions and to provide suitable environmentally friendly solvents in a process for the manufacture of valsartan.

Accordingly, the present invention relates to an improved method for the preparation of valsartan having an enantiomeric purity of at least 99.8% or more.

In one embodiment of the present invention, the intermediate compound of Formula II is prepared by (1) reacting a compound of Formula IIa with compound of Formula IIb in a heterogeneous solvent system/medium containing a mixture of water and an organic solvent, in presence of an inorganic base; and (2) isolating the product (Formula II) as a hydrochloride salt or free base. The said reaction may be optionally performed in presence of a catalyst.

In another embodiment of the present invention, the compound II is acylated with valeroyl chloride in presence of an organic base characterized in solvents selected from non-polar solvents like toluene, xylene etc. The reaction proceeds substantially to completion (e.g. by conversion of at least 99.8% of the starting compound II) in a short period of time.

In yet another embodiment of the present invention the benzyl valsartan intermediate (Formula IV) substantially free of the organotin impurity is provided by (1) first reacting Compound III with tributyl tin azide in toluene or xylene; (2) followed by purifying the isolated crude benzyl valsartan (Formula IV) contaminated with organotin impurity from a first solvent that is a ternary solvent mixture; and (3) followed by crystallizing from a second solvent, such as a non-polar organic solvent or a polar aprotic solvent or mixtures thereof. The first solvent is characterized by combination of a hydrophilic organic solvent selected from C₁ to C₄ alcohol, a non-polar organic solvent like hexane or toluene or the like and water

In a further aspect, the present invention provides a process for debenzylation of benzyl valsartan substantially free of organotin impurity using palladium carbon characterized by a significantly lower catalyst loading relative to the prior art processes, at ambient temperature, in a hydrophilic organic solvent selected from C₁ to C₄ alcohol; followed by crystallization of valsartan of enantiomeric purity of at least 99.8% from solvents mixture, such as ethyl acetate and hexane or ethyl acetate and ether, at a temperature below 60° C.

### DETAILED DESCRIPTION OF THE INVENTION

The invention will now be described in detail in connection with certain preferred and optional embodiments, so that various aspects thereof may be more fully understood and appreciated. The present invention thus provides a process for preparation of valsartan and its intermediates in substantially pure enantiomeric form.

In the first step, the compound of the general Formula IIa is reacted with compound of general formula IIb in presence of an inorganic base and optionally in presence of a catalyst. In a preferred embodiment of the present invention the above reaction step is carried out in a heterogeneous solvent mixture comprising water and a non-polar hydrocarbon solvent.

Formula IIa is as follows: 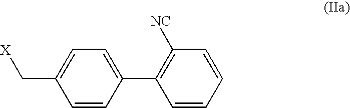
where X is any halogen atom; preferably bromine.

Formula IIb is as follows: 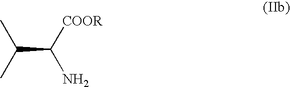
where R represents any carboxyl group, however, the preferred carboxyl group is a methyl or benzyl group. In the reaction the compound IIb may be used as its acid salt, preferably a hydrochloric acid salt or a p-toluene sulphonic acid salt, and more preferably a p-toluene sulphonic acid salt. If an acid salt is employed in the reaction, an excess amount of base is used, which is required to neutralize the salt to the free compound IIb.

The heterogeneous solvent mixture comprises water and an organic solvent, preferably a hydrocarbon solvent, such as toluene or xylene. The organic solvent and water mixture comprises, based on the total volume of the solvent mixture, from about 40% to 80% of non-polar organic solvent and about 60% to 20% of water. Preferably, the reaction is carried out in a mixture of non-polar hydrocarbon solvent and water comprising, based on the total volume of the solvent mixture, from about 40% to 60% of non-polar hydrocarbon solvent and about 60% to 40% of water.

As described above the preferred non-polar hydrocarbon solvents may be, but not limited to, C₆-C₉ aromatic hydrocarbon, C₅-C₈ aliphatic hydrocarbon, and C₅-C₈ alicyclic hydrocarbon. The preferred non-polar solvent may be, but is not limited to, toluene, xylene, hexane, cyclohexane, or mixtures thereof. The most preferred non-polar solvent is toluene. Other hydrocarbons useful in the practice of the present invention may also be used and are apparent those skilled in the art.

The basic material used in the reaction is an inorganic base such as carbonate or bicarbonate salt of an alkali metal. The preferred alkali metal salt is sodium carbonate, potassium carbonate, or sodium bicarbonate.

The reaction may optionally be carried out in the presence of a catalyst. Preferred catalyst may be, but not limited to, potassium iodide; sodium iodide; a phase-transfer catalyst, such as tetrabutyl ammonium bromide or tetrabutyl ammonium chloride; or combinations thereof, such as a phase-transfer catalyst and sodium/potassium iodide.

The reaction may be carried out at a temperature ranging from an ambient temperature to the reflux temperature of the reaction mixture. More preferably, heat is added to effect the completion of reaction at a temperature of about 40° C. to 60° C. and the reaction completes in a period of about 20 hours to 30 hours.

On completion of the reaction, the intermediate compound II is isolated from the reaction mixture by phase separation and water washing, and acidification of the organic layer. The reaction product (S)-N-[(2′-cyanobiphenyl-4-yl)methyl]-(L)-valine benzyl ester of formula II, gets separated out in the form of its hydrochloride salt after acidification of the reaction mixture using hydrogen chloride (gaseous hydrochloric acid or aqueous hydrochloric acid) to a pH of 1-4. Other acids may also be used, including, but not limited to, commonly used mineral acids or organic acids, such as methane sulphonic acid, benzene sulphonic acid, or the like.

The precipitated (S)-N-[(2′-cyanobiphenyl-4-yl)methyl]-(L)-valine benzyl ester hydrochloride shows a purity above 95% with an yield of about 85%. The (S)-N-[(2′-cyanobiphenyl-4-yl)methyl]-(L)-valine benzyl ester hydrochloride is used as such in subsequent reaction or may be isolated as its free base by neutralization procedures known in the art.

In a second step of the present invention, the (S)-N-[(2′-cyanobiphenyl-4-yl)methyl]-(L)-valine benzyl ester (Formula II) or its hydrochloride salt is reacted with the compound of formula IIc in presence of a base, such as diisopropylethylamine, to form a compound of Formula III.

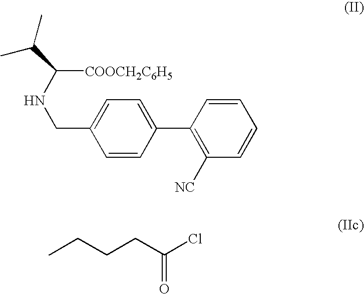

According to the present invention the above process step, reaction of (S)-N-[(2′-cyano- biphenyl-4-yl)methyl]-(L)-valine benzyl ester (Formula II) or its hydrochloride salt with compound llc, is carried out in a non-polar hydrocarbon solvent. The preferred non-polar solvent is selected from C₆-C₉ aromatic hydrocarbon, C5-C₈ aliphatic hydrocarbon, and C₅-C₈ alicyclic hydrocarbon, with toluene being the preferred.

The reaction is carried out at a temperature between 0°C. and the reflux temperature, preferably between 20° C. to the reflux temperature of the solvent. Most preferably, the reaction is carried out at about 25° C.-35° C., and completes in a span of about 1 to 5 hours.

In the third step, the cyano group in compound III is converted into tetrazole ring system by reacting compound III with tributyltin azide to produce the penultimate intermediate of valsartan called benzyl valsartan (Formula IV). Preferably, the tributyltin azide is added in three lots; toluene is used as solvent; and the reaction is effected at reflux temperature.

In a preferred embodiment of the present invention, the stage III product (Formula III) after water washings is directly employed in the tetrazole formation when the reaction solvent is toluene or xylene. After the completion of tetrazole formation, the benzyl valsartan is released from the tributyl tin complex by passing dry hydrochloric acid gas through the reaction mixture, decanting off the toluene, dissolving the residue in a water immiscible solvent, and washing the resulting solution (formed in the water immiscible solvent) with water until free of acid. The compound of Formula IV is isolated as oil in crude form after evaporation of the water immiscible solvent.

Preferably, the second step and third step are carried out in situ without isolating the product of second step prior to performing the third step.

The present invention also provides a purification method for effective removal of the organotin impurity from benzyl valsartan of the formula (IV). The purification process includes (1) a first crystallization of benzyl valsartan from a ternary solvent mixture comprising a hydrophilic organic solvent, a non-polar organic solvent, and water; and (2) a second crystallization from a polar aprotic solvent, a non-polar organic solvent, or mixtures thereof. Preferably, the second crystallization solvent is a binary solvent mixture comprising a polar aprotic solvent and a non-polar organic solvent.

The hydrophilic organic solvent used in the ternary solvent mixture in the first crystalization step is selected from C₁ to C₄ alcohols, preferably isopropanol. The non-polar organic solvents are selected from hydrocarbon solvents, such as hexane, toluene, cyclohexane, or the like. The composition of the ternary mixture is as follows: the ratio of hydrophilic organic solvent to inert non-polar organic solvent is in the range of about 1:0.5 to 1:100 parts by weight, preferably about 1:0.5 to 1:25 parts by weight, and more preferably about 1:0.5 to 1:10 parts by weight, on the basis of 1 part by weight of compound of Formula IV. The ratio of water to combination of hydrophilic organic solvent and inert non-polar organic solvent is about 0.5:1 to 10:1 parts by weight, and preferably about 0.5:1 to 5:1 parts by weight, on the basis of 1 part by weight of compound of formula IV.

Preferably, water is added to a pre-formed solution of compound of Formula IV (benzyl valsartan) in a mixture of a hydrophilic organic solvent and a non-polar organic solvent between 0° C. to reflux temperature. More preferably, the addition is carried out between 25° C. -35° C. and the benzyl valsartan (Formula IV) precipitates out of the solution. The precipitated compound (Formula IV) is isolated by filtration or other conventional means at ambient temperature or at 0° C.

According to the present invention, benzyl valsartan (Formula IV) is further purified from a second solvent, preferably a non-polar organic solvent, a polar aprotic solvent, or mixtures thereof, to obtain benzyl valsartan substantially free of organotin impurity in high enantiomeric purity.

The non-polar organic solvent may be, but not limited to, C₆-C₉ aromatic hydrocarbon, C₅-C₈ aliphatic hydrocarbon, C₅-C₈ alicyclic hydrocarbon, or mixtures thereof. The preferred non-polar organic solvents are selected from hydrocarbon solvents, such as hexane, toluene, or the like. The preferred inert polar aprotic solvent is ethyl acetate.

In the process of purification, the non-polar organic solvent is added to a solution of benzyl valsartan (Formula IV) in a polar aprotic solvent between 0° C. and reflux temperature. Preferably, the addition of the non-polar organic solvent is carried out between 25° C. -35° C. to precipitate the benzyl valsartan substantially free of the organotin impurity. The tin content of the resultant benzyl valsartan is preferably less than 5000 ppm, and more preferably the benzyl valsartan is substantially free of any detectable tin content as measured by atomic emission spectroscopy.

The ratio of the polar aprotic solvent to the non-polar organic solvent is about 1:0 to 1:100 parts by weight, preferably about 1:0 to 1:50 parts by weight, and more preferably about 1:0 to 1:25 parts by weight, on the basis of 1 part by weight of compound of Formula IV. The precipitated benzyl valsartan (Formula IV) can be separated from the mixture by conventional means, such as filtration, and optionally, dried at a temperature below 60° C.

The benzyl valsartan (Formula IV) obtained above is subjected to hydrogenation in presence of palladium-charcoal to obtain valsartan. The present invention is characterized by a low loading of costly palladium-charcoal catalyst in the debenzylation of benzyl valsartan substantially free of organotin impurity. In the method of the prior art, the organotin impurity poisons the catalyst, and significantly reduces its activity which necessitates a heavy loading of costly palladium-charcoal catalyst. The present invention has reduced the palladium-charcoal quantity by about 70% (16 times reduction on a wt/wt basis) with respect to the prior art processes.

After the completion of the reaction, the product is filtered, concentrated, and prepared as solution of valsartan (Formula I) in an aqueous basic solution. The aqueous basic solution may be that of an alkali metal or alkaline earth metal salt, such as sodium carbonate, sodium bicarbonate, potassium carbonate, sodium hydroxide, or potassium hydroxide. The aqueous layer is washed with a chlorinated solvent, such as methylene dichloride or ethylene dichloride. The aqueous solution is then rendered acidic using an aqueous acidic solution, such as hydrochloric acid, sulfuric acid, or acetic acid to precipitate the valsartan. The precipitated valsartan (in the acidified water) is then extracted using an organic solvent, such as ethyl acetate, isobutyl methyl ketone, or methyl propyl ketone, where the valsartan dissolves in the organic phase. The organic phase is concentrated under reduced pressure to dryness. The residue is suspended and slurry washed with hydrocarbon solvent, such as toluene, hexane, or cyclohexane; a combination of hydrocarbon solvent with ethyl acetate or diisopropyl ether; or mistures thereof. The valsartan (Formula I) can then be separated from the solvent mixture by decanting, filtering, centrifuging, other similar processing methods of isolation of solids from liquids known to those skilled in the art, or any combination of such separation methods; and optionally followed by a wash with water.

Valsartan after acidification and extraction in the above solvent can also be isolated by slurryfication, at a temperature below about 60° C., from a premix solvent mixtures, such as ethyl acetate-hexane or ethyl acetate-diisopropylether, optionally in presence of water. Valsartan can be separated from the mixture by decanting, filtering, centrifuging, using other similar processing methods of isolation of solids from liquids known to those skilled in the art, or any combination of such separation methods; and optionally followed by a wash with water.

Isolated valsartan is dried, at a temperature below 60° C., by air drying, vacuum drying, fluidized bed drying, other similar drying methods of solids known to those skilled in the art, or any combination of such drying methods.

The temperature of reaction as well as isolation and drying is crucial for obtaining high enantiomeric excess, as at higher temperature, valsartan tends to racemize. The valsartan obtained by following the process of the present invention has a purity of at least 99.7% for the S-isomer, and less than about 0.10% for the R-isomer. This drastic yield and purity improvement caused by the present invention thus leads to an efficient and commercially acceptable synthetic process for the preparation of valsartan. This higher enantiomeric purity and yield starting from compound of Formula II constitutes a considerable technical advance with respect to the processes of the prior art.

The invention is explained in more detail in the following working examples. The examples, which illustrate improvement in the method according to the invention, have a purely illustrative character and do not limit the extent of the invention in any respect.

### Example 1

### (S)-N-[(2′-cyanobiphenyl4-yl)methyl]-(L)-valine benzyl ester hydrochloride (Formula II)

4-bromomethyl-2′-cyanobiphenyl (15 g, 0.055 mol), L-valine benzyl ester tosylate (20.9 g, 0.055 mol), potassium carbonate (21 g, 0.152 mol), and potassium iodide (0.21 g) were heated to 50-55° C. in toluene (63 mL) and water (63 mL) for 25 hours. After cooling, the two phases separated. The organic layer was water washed (2×50 mL) and acidified with hydrochloric acid to pH 1-2; upon which, (S)-N-[(2′-cyanobiphenyl-4-yl)methyl]-(L)-valine benzyl ester hydrochloride crystallized. Precipitated solids were filtered, washed with toluene and dried to give 17.9 g (75% yield) of (S)-N-[(2′-cyanobiphenyl-4-yl)methyl]-(L)-valine benzyl ester hydrochloride having a purity of 97% as measured by HPLC area percent.

### Example 2

### (S)-N-[(2′-cyanobiphenyl-4-yl)methyl]-(L)-valine benzyl ester hydrochloride (Formula II)

4-bromomethyl-2′-cyanobiphenyl (15 g, 0.055 mol), L-valine benzyl ester tosylate (20.9 g, 0.055 mol), potassium carbonate (21 g, 0.152 mol), and tetrabutyl ammonium bromide (0.21 g) were heated to 50-55° C. in xylene (63 mL) and water (63 mL) for 25 hours. After cooling, the two phases separated. The organic layer was water washed and acidified with hydrochloric acid to pH 1-2; upon which, (S)-N-[(2′-cyanobiphenyl-4-yl)methyl]-(L)-valine benzyl ester hydrochloride precipitated. The obtained solids were filtered, washed with toluene and dried to give 17.4 gms (73% yield) of (S)-N-[(2′-cyanobiphenyl-4-yl)methyl]-(L)-valine benzyl ester hydrochloride having a purity of 97% as measured by HPLC area percent.

### Example 3

### (S)-N-[(2′-cyanobiphenyl-4-yl)methyl]-(L)-valine benzyl ester hydrochloride (Formula II)

4-bromomethyl-2′-cyanobiphenyl (4.2 kg, 0.0154 Kmol), L-valine benzyl ester tosylate (5.85 kg, 0.0154 Kmol), potassium carbonate (5.88 kg, 0.0426 Kmol), tetrabutyl ammonium bromide (0.059 kg) and potassium iodide (0.059 kg) were heated to 50-55° C. in toluene (17.6 L) and water (17.6 L) for 25 hours. After cooling, the two phases separated. The organic layer was water washed (2×14 L) and acidified with hydrochloric acid to pH 1-2; upon which, the (S)-N-[(2′-cyanobiphenyl-4-yl)methyl]-(L)-valine benzyl ester hydrochloride precipitated which was filtered, washed with toluene and dried to give 6.05 kgs (90% yield) of (S)-N-[(2′-cyanobiphenyl-4-yl)methyl]-(L)-valine benzyl ester hydrochloride having a purity of 97% as measured by HPLC area percent.

### Example 4

### (S)-N-[(2′-cyanobiphenyl-4-yl)methyl]-N-valeroyl-(L)-valine benzyl ester (Formula III)

To sodium bicarbonate (65 g, 0.774 mol) in water (800 mL) and toluene (400 mL) was added (S)-N-[(2′-cyanobiphenyl-4-yl)methyl]-(L)-valine benzyl ester hydrochloride (210 g, 0.483 mol) at 25° C. The mixture was stirred for an hour; after which, the layers separated. The toluene layer was then washed with a brine solution and dried over sodium sulfate. To the dried toluene layer containing, (S)-N-[(2′-cyano biphenyl-4-yl)methyl]-(L)-valine benzyl ester, N,N-diisopropylethylamine (101.8 g, 0.786 mol) was added at 25° C. and cooled to 20° C. Valeroyl chloride (80 g, 0.664 mol) was then added over a period of about 30 minutes. After the completion of the reaction by thin layer chromatography (TLC) check, 50 mL water was added and further stirred for 30 minutes. The layers were separated; and IN hydrochloric acid solution was added to the organic layer at 0°C. until the pH reaches about 1-3. The acidified mixture was stirred for 30 minutes; and the layers separated. 10% sodium bicarbonate solution was added to the organic layer until the pH of reaction mass reaches about 7-8. The layers then separated; and the organic layer was washed with 200 mL water, and then with 200 mL brine solution. The organic layer was dried, solvent distilled out under reduced pressure to obtain 228 g (98% yield) of (S)-N-[(2′-cyanobiphenyl-4-yl)methyl]-N-valeroyl-(L)-valine benzyl ester as a brownish oil having a purity of 96% as measured by HPLC area percent.

### Example 5

### (S)-N-(1-benzyloxycarbonyl-2-methyl-prop-1-yl)-N-pentanoyl-N-[2′-(1H-tetrazol-5-yl)-biphenyl-4-ylmethyl]-amine (Formula IV)

To a mixture of sodium bicarbonate (65 g, 0.774 mol), water (800 mL) and toluene (400 mL) was added (S)-N-[(2′-cyanobiphenyl-4-yl)methyl]-(L)-valine benzyl ester hydrochloride (210 g, 0.483 mol) at 25° C. and stirred for an hour. The layers were separated; and the toluene layer was dried over sodium sulfate. N,N-diisopropylethylanine (101.8 gm, 0.786 mol) was added to the dried toluene layer, containing (S)-N-[(2′-cyano biphenyl-4-yl)methyl]-(L)-valine benzyl ester, at 25° C. It was then cooled down to 15° C.; and valeryl chloride (80 g, 0.664 mol) was added over a period of about 60 minutes. Reaction progress monitored by TLC, on completion of reaction 100 mL water was added and further stirred for 30 minutes. The organic layer separated out and washed first with 1N hydrochloric acid solution until the pH was about 1-3, and then washed with 10% aqueous sodium bicarbonate solution until the pH of the washings reaches 7-8 respectively. The toluene layer was further washed with 200 mL water and then with 200 mL brine solution. The organic layer was subsequently dried using sodium sulfate. The dried organic layer was further used as such for tetrazole formation.

To the above dried toluene layer was added sodium azide (70 g, 1.08 mol) and tributyl tin chloride (350 g, 1.07 mol), and heated to reflux. After about 20 hrs, a second lot of sodium azide (10 g, 0.154 mol) and tributyl tin chloride (50 g, 0.154 mol) was added. The reflux continued for 20-24 hrs. After the completion of reaction by TLC check, the reaction mass was cooled to about 15° C. Dry hydrochloric acid gas was then passed through the toluene layer until the decolorization of toluene was observed. The toluene layer was decanted; and the residue was washed with 100 mL of toluene. The residue was then partitioned between methylene chloride and a 10% sodium bicarbonate solution, and stirred at 25-30° C. for 30 minutes. The layers then separated; and the organic layer was washed with a brine solution and dried over sodium sulfate. The solvent was distilled off under reduced pressure to obtain 240 g of (S)-N-(1-benzyloxycarbonyl-2-methyl-prop-1-yl)-N-pentanoyl-N-[2 ′-(1H-tetrazol-5-yl)-biphenyl-4-ylmethyl]-amine [benzyl valsartan] as a brownish oil having HPLC assay purity 91%.

### Example 6

### Purification of (S)-N-(1-benzyloxycarbonyl-2-methyl-prop-1-yl)-N-pentanoyl-N-[2′-(1H-tetrazol-5-yl)-biphenyl-4-ylmethyl]-amine (Benzyl valsartan)

23 g of the crude (S)-N-(1-benzyloxycarbonyl-2-methyl-prop-1-yl)-N-pentanoyl-N-[2′-(1H-tetrazol-5-yl)-biphenyl-4-ylmethyl]-amine oil obtained in Example 5 was taken in 40 mL isopropyl alcohol (IPA) and heated to obtain a clear solution. To the reaction mass was added 200 mL hexane. It was further cooled to about 10-20° C.; and 250 ml water was added in about 60 minutes. The mixture was stirred at 25-30° C. for 60 minutes. It was filtered, washed with water and dried to give 18 gris (78% yield) of (S)-N-(1-benzyloxycarbonyl-2-methyl-prop-1-yl)-N-pentanoyl-N-[2 ′-(1H-tetrazol-5-yl)-biphenyl-4-ylmethyl]-amine having a purity of 94% as measure by HPLC assay and a melting point (MP) of 106-108° C.

To 5 g of the above solid was added to 15 mL toluene and heated to obtain a clear solution. 10 mL hexane was then added and cooled to ambient temperature; upon which (S)-N-(1-benzyloxycarbonyl-2-methyl-prop-1-yl)-N-pentanoyl-N-[2′-(1H-tetrazol-5-yl)-biphenyl-4-ylmethyl]-amine crystallized. It was filtered, washed with hexane and dried to give 4.5 gms (90% yield) of (S)-N-(1-benzyloxycarbonyl-2-methyl-prop-1-yl)-N-pentanoyl-N-[2′-(lH-tetrazol-5-yl)-biphenyl-4-ylmethyl]-amine having purity of 100.46% as measured by HPLC assay with no detectable tin content (the analysis was performed by HPLC assay, which can be over 100% (assay limit is generally about 98-102%)) and a MP of 115.9-116° C.

### Example 8

### Purification of Benzyl valsartan (Formula IV)

1.53 kgs of the crude (S)-N-(1-benzyloxycarbonyl-2-methyl-prop-1-yl)-N-pentanoyl-N-[2′-(1H-tetrazol-5-yl)-biphenyl-4-ylmethyl]-amine (Formula IV) oil prepared according to the example 5 was taken in 2.67 L of isopropyl alcohol and heated to obtain a clear solution. To the reaction mass was added 13.3 L hexane. It was further cooled to about 10-20° C.; and 16.67 L water was added in about 60 minutes. The mixture was stirred at 25-30° C. for 60 minutes. Precipitate was filtered, washed with water and dried to give 1.2 kgs (78% yield) of (S)-N-(1-benzyloxycarbonyl-2-methyl-prop-1-yl)-N-pentanoyl-N-[2′-(1H-tetrazol-5-yl)-biphenyl-4-ylmethyl]-amine having purity93-96%(HPLC assay). The product has melting point 106-108° C.;

1 kg of the above obtained solid was added to 2 L ethyl acetate and heated to obtain a clear solution. 4 L hexane was then added and cooled to 25-30° C.; upon which, (S)-N-(1-benzyloxycarbonyl-2-methyl-prop-1-yl)-N-pentanoyl-N-[2 ′-(1H-tetrazol-5-yl)-biphenyl-4-ylmethyl]-amine crystallized out. The precipitate was filtered, washed with hexane and dried to give 3.5 kgs (70% yield) of (S)-N-(i-benzyloxycarbonyl-2-methyl-prop-1-yl)-N-pentanoyl-N-[2′-(1H-tetrazol-5-yl)-biphenyl-4-ylmethyl]-amine having purity 100.15% (HPLC assay) with no detectable tin content (analyzed by atomic emission spectroscopy). The product melts at 115.2-115.8° C.

### Example 9

### (S)-N-(1-Carboxy-2-methyl-prop-1-yl)-N-pentanoyl-N-[2′-(1H-tetrazol-5-yl)-biphenyl-4-ylmethyl]-amine (Valsartan)

A solution of 50 g (S)-N-(1-benzyloxycarbonyl-2-methyl-prop-1-yl)-N-pentanoyl-N-[2′-(1H-tetrazol-5-yl)bi-phenyl-4-ylmethyl]-amine in 500 mL methanol was hydrogenated at room temperature with 2.5 g (5% loading) of 5% Pd—C until the completion of reaction. After reaction catalyst was filtered out and crude valsartan was obtained by evaporation of the solvent. It was then partitioned between 500 mL of 10% sodium bicarbonate solution and 200 mL MDC [dichloromethane]. The aqueous phase was separated and rendered acidic. Valsartan was isolated by extraction with ethyl acetate, and crystallization from a mixture of ethyl acetate and hexane. The precipitated valsartan was filtered, washed with hexane and dried to give 34 gms (82% yield) of Valsartan. Purity of valsartan obtained was 99.8% and R-isomer 0.07% as measured by HPLC area percent.

### Example 10

### (S)-N-(1-Carboxy-2-methyl-prop-1-yl)-N-pentanoyl-N-[2′-(1H-tetrazol-5-yl)-biphenyl-4-ylmethyl]-amine (Valsartan)

A solution of 50 g (S)-N-(1-benzyloxycarbonyl-2-methyl-prop-1-yl)-N-pentanoyl-N-[2′-(1H-tetrazol-5-yl)bi-phenyl-4-ylmethyl]-amine in 300 mL methanol was hydrogenated at room temperature with 1.25 g (2.5% loading) of 5% Pd—C and further proceeded analogously to example 9. Valsartan was crystallized from a mixture of ethyl acetate and di-isopropyl ether, filtered, washed with di-isopropyl ether and dried to give 29 gms of Valsartan (Yield-70%). Purity of valsartan was 99.83% and R-isomer 0.03% as measured by HPLC area percent.

### Example 11

### Valsartan

A solution of 50 g (S)-N-(1-benzyloxycarbonyl-2-methyl-prop-1-yl)-N-pentanoyl-N-[2′-(1H-tetrazol-5-yl)bi-phenyl-4-ylmethyl]-amine in 500 mL methanol was hydrogenated at room temperature with 1.25 g (2.5% loading) of 5% Pd—C until the completion of reaction. The crude valsartan was obtained by filtration and evaporation of the solvent. The residue was partitioned between 500 mL of 10% sodium bicarbonate solution and 200 mL MDC. The aqueous phase was separated and rendered acidic. Valsartan was isolated by extraction with ethyl acetate. The organic phase was distilled under reduced pressure to dryness. The residue was then suspended in a mixture of ethyl acetate and hexane at ambient temperature, filtered, Clashed with water, and dried to give 34 grns of Valsartan (yield of 82%). Purity of Valsartan obtained was 99.8% and R-isomer 0.05% by HPLC analysis.

### Example 12

### Valsartan

A solution of 50 g (S)-N-(1-benzyloxycarbonyl-2-methyl-prop-1-yl)-N-pentanoyl-N-[2′-(1H-tetrazol-5-yl)bi-phenyl-4-ylmethyl]-amine in 500 mL methanol was hydrogenated at room temperature with 2.5 gm (5% loading) of 5% Pd—C until the completion of reaction. The crude valsartan was obtained by filtration and evaporation of the solvent, and was partitioned between 500 mL of 10% sodium bicarbonate solution and 200 mL MDC. The aqueous phase was separated and rendered acidic. Valsartan was isolated by extraction with ethyl acetate. The organic phase was distilled under reduced pressure to dryness, slurried in a mixture of ethyl acetate:hexane:water (ratio 2:5:1), filtered, and dried to obain 34 gm (82% yield) of valsartan. Purity of valsartan obatinaed was 99.8% and R-isomer 0.02% as measured by HPLC analysis.

### Example 13

### (S)-N-[(2′-cyanobiphenyl4-yl)methyl]-N-valeroyl-(L)-valine benzyl ester (Formula III) per U.S. Pat. No. 5,399,578

6.2 g (15.5 mmol) of (S)-N-[(2′-cyanobiphenyl-4-yl)methyl]-(L)-valine benzyl ester and 8.0 mL N,N-diisopropylethylamine, dissolved in 50 ml of methylene chloride, are treated with 2.3 mL of valeryl chloride with stirring. The reaction mixture was stirred at room temperature for 20-25 hours until the starting amine can no longer be detected by TLC. After evaporating in a water jet vacuum, the reaction mixture was partitioned between 82 mL water and 826 mL ethyl acetate. The organic phase was washed successively with 40 mL each of 2N hydrochloric acid, saturated NaHCO₃ solution, and brine; dried over magnesium sulfate; and evaporated in vacuum. The title compound obtained as brown oil with a purity of 72% as measured by HPLC area percent.

### Example 14

### (S)-N-(1-benzyloxycarbonyl-2-methyl-prop-1-yl)-N-pentanoyl-N-[2′-(1H-tetrazol-5-yl)-biphenyl-4-ylmethyl]-amine (Benzyl valsartan) per U.S. Pat. No. 5,399,578

6.6 g (13.6 mmol) of (S)-N-[(2′-cyanobiphenyl-4-yl)methyl]-N-valeroyl-(L)-valine benzyl ester and 6.0 g (18 mmol) of tributyltin azide, in 75 mL of o-xylene, were heated to boiling with stirring for 48 hours. After 24 hours, an additional 2.0 g of tributyltin azide was added. After cooling, the solution was diluted with about 125 mL of toluene, treated with 110 mL 1N potassium hydroxide solution, and stirred for 20 minutes. The aqueous phase was separated and rendered acidic with IN hydrochloric acid solution. The product was extracted in ethyl acetate. The title compound after evaporation of solvent was obtained as oil with a purity of 86-88% as analyzed by HPLC assay.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

What is claimed is:

1. A process for preparing valsartan of Formula I 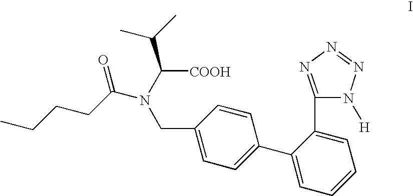
comprising the steps of:
a) purifying intermediate benzyl valsartan of Formula IV 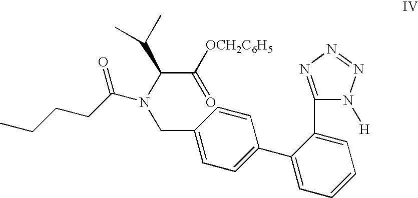
by (i) crystallizing said benzyl valsartan from a first solvent which is a ternary mixture comprising a hydrophilic solvent, a non-polar protic solvent, and water; (ii) recovering benzyl valsartan from said ternary mixture; (iii) crystallizing benzylvalsartan from a second solvent comprising a non-polar aprotic solvent, a polar aprotic solvent, or a mixture thereof; and (iv) recovering benzyl valsartan substantially free of organotin impurity; and
b) converting said benzyl valsartan of step (a) into valsartan

2. The process of claim 1, wherein the hydrophilic solvent is selected from the group consisting of C1-C4 alcohols, and the non-polar protic solvents is selected from the group consisting of C₆-C₉ aromatic hydrocarbon, C₅-C₈ aliphatic hydrocarbon, and C₅-C₈ alicyclic hydrocarbon.

3. The process of claim 1, wherein said ternary solvent mixture has the hydrophilic organic solvent to the non-polar protic solvent in a ratio of about 1:0.5 to 1:30, and the hydrophilic organic solvent and non-polar organic solvent to water in a ratio of about 1:0.5 to 1:10 parts by weight relative to the benzyl valsartan

4. The process of claim 1, wherein the hydrophilic solvent is isopropanol.

5. The process of claim 1, wherein the non-polar protic solvent is hexane or toluene.

6. The process of claim 1, wherein the second solvent has a polar aprotic solvent to non-polar aprotic solvent in a ratio of about 1:0 to 1:50 parts by weight relative to the benzyl valsartan

7. The process of claim 1, wherein the second solvent is selected from ethyl acetate, hexane, toluene, or mixtures thereof.

8. The process of claim 1, wherein the second crystallization solvent is a binary mixture comprising a non-polar aprotic solvent and a polar aprotic solvent.

9. The process of claim 8, wherein the binary mixture is a combination of ethyl acetate and hydrocarbon solvent.

10. The process as claimed in claim 1, wherein step (b) comprises
i) hydrogenation of benzyl valsartan obtained from step (a) in presence of palladium carbon catalyst in a quantity less than 10% wt/wt relative to benzyl valsartan; and
ii) recovering the valsartan.

11. The process of claim 10, wherein the catalyst quantity is 2.5-5% wt/wt relative to the benzylvalsartan.

12. The process of claim 10, further comprising the step of drying the valsartan at a temperature less than 60° C.

13. The process of claim 1, wherein the benzyl valsartan obtained from step a) has less than about 1000 ppm (parts per million) of organotin impurity as measured by atomic emission spectroscopy and less than 0.1% of benzyl (R)-N-(1-Carboxy-2-methyl-prop-1-yl)-N-pentanoyl-N-[2′-(1H-tetrazol-5-yl)-biphenyl-4-ylmethyl]-amine isomer as measured by HPLC area percent.

14. The process of claim 1, wherein the valsartan has an enantiomeric purity greater than 99.5%.

15. The process of claim 1, wherein the valsartan has less than about 0.1% (R)-N-(1-Carboxy-2-methyl-prop-1-yl)-N-pentanoyl-N-[2′-(1H-tetrazol-5-yl)-biphenyl-4-ylmethyl]-amine isomer as measured by HPLC area percent.

16. A process for preparing valsartan of Formula I 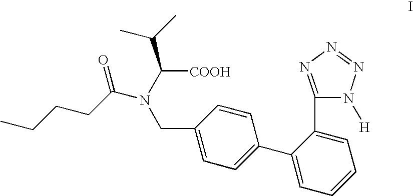
comprising the steps of:
a) providing benzyl valsartan of Formula IV 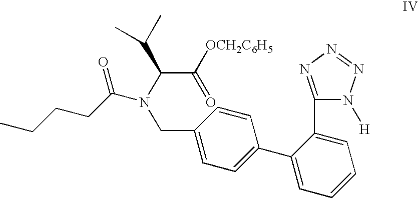
by reacting compound of Formula III 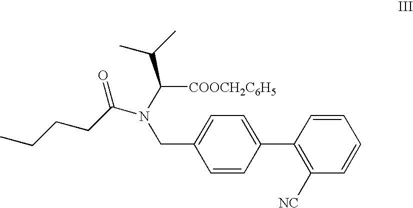
with a tributyl tin azide complex in presence of a hydrocarbon solvent;
b) purifying said benzyl valsartan by (i) crystallizing from a first solvent which is a ternary mixture comprising a hydrophilic solvent, a non-polar solvent, and water; (ii) recovering the benzyl valsartan from said first solvent; (iii) crystallizing the benzyl valsartan from a second solvent which is a binary mixture comprising non-polar aprotic solvent and polar aprotic solvent; and (iv) recovering the benzyl valsartan substantially free of organotin impurity; and
c) converting said benzyl valsartan recovered in step (b) into valsartan using palladium carbon catalyst in a quantity less than 10 wt/wt relative to benzyl valsartan.

17. The process of claim 16, wherein the tributyl tin azide is prepared in situ from tributyl tin chloride and sodium azide.

18. The process of claim 16, wherein the ternary solvent mixture comprises of isopropyl alcohol, hexane, and water.

19. The process of claim 16, wherein the second crystallization solvent in step is a binary mixture comprising ethyl acetate and hexane.

20. The process of claim 16, wherein the benzyl valsartan obtained from step (b) is of 99.5% purity.

21. A process for preparing valsartan of Formula I 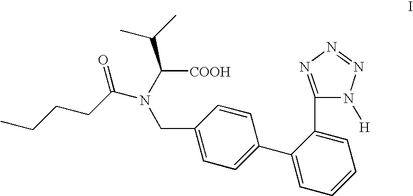
comprising the steps of:
a) reacting 4-bromomethyl-2′-cyanobiphenyl with L-valine benzyl ester or its acid salt in a heterogeneous solvent mixture comprising water and a non-polar hydrocarbon solvent, in presence of a inorganic base to obtain a compound of Formula II 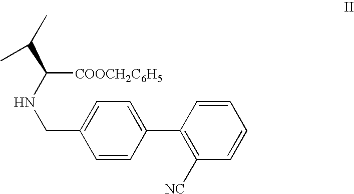
b) reacting the compound of Formula II with valeroyl chloride in presence of diisopropylethylamine in a solvent selected from toluene or hexane to produce the compound of Formula III 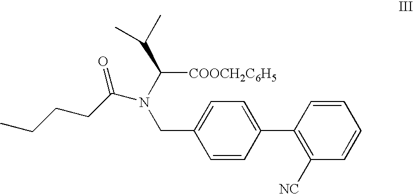
c) providing benzylvalsartan of Formula IV 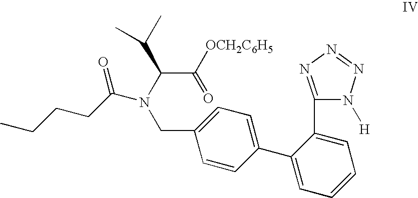
from the compound of Formula III using an tributyltinazide;
d) purifying said benzyl valsartan by (i) crystallizing the benzyl valsartan from a first solvent which is a ternary mixture comprising a hydrophilic solvent, a non-polar solvent, and water; (ii) recovering the benzyl valsartan from said ternary mixture; (iii) crystallizing the benzyl valsartan from a second solvent which is a binary mixture comprising ethyl acetate and hexane; and (iii) recovering the benzyl valsartan substantially free of tributyl tin impurity;
e) converting said benzyl valsartan purified from step d) into valsartan using palladium carbon; and
f) recovering the valsartan produced in step (e), wherein said valsartan has less than 0.1% (R)-N-(1-Carboxy-2-methyl-prop-1-yl)-N-pentanoyl-N-[2′-(1H-tetrazol-5-yl)-biphenyl-4-ylmethyl]-amine isomer.

22. The process of claim 21, wherein step (a) is carried out in presence of a catalyst selected from the group consisting of sodium iodide, potassium iodide, a phase-transfer catalyst, and combinations thereof.

23. A pharmaceutical composition comprising valsartan obtained from the process of claim 1 in a pharmaceutically acceptable form.

24. A pharmaceutical composition of claim 23, further comprising a diuretic agent

25. The pharmaceutical composition of claim 24, wherein the diuretic agent is hydrochlorothiazide or chlorthalidone.
